(19) United States
(12) Patent Application Publication
(10) Pub. No.: US 2023/0044896 A1
(43) Pub. Date: Feb. 9, 2023

(54) REMOVABLE ATTACHMENT SYSTEM ON A MAST FOR A NON-IMPLANTABLE MEDICAL DEVICE

(71) Applicant: AXESS VISION TECHNOLOGY, JOUE-LES-TOURS (FR)

(72) Inventors: Emmanuel HALLAUER, SACHE (FR); Philippe LE ROUX, TOURS (FR); Pascal COCHARD, DIERRE (FR)

(21) Appl. No.: 17/793,067

(22) PCT Filed: Dec. 22, 2020
(86) PCT No.: PCT/FR2020/052595

§ 371 Date: Jul. 15, 2022

(30) Foreign Application Priority Data
Jan. 17, 2020 (FR) FR2000447

(51) Int. Cl. A61B 1/00; A61B 50/13; H04N 5/225

(52) U.S. Cl. CPC A61B 1/00048; A61B 50/13; H04N 5/2252; H04N 5/225251; H04N 2005/2255

(57) ABSTRACT

The disclosure relates to a system for attaching a medical device on a mast, the attachment system including a body equipped with the medical device and from which protrude face-to-face two jaws together delimiting an open engagement housing for the mast ending, at the level of the body, in a bottom which gradually decreases along a frustoconical portion to adapt to masts of different diameters, both jaws being externally threaded by a thread including a tapped ring and a tubular support skirt for the jaws, this tubular skirt extending coaxially inside the tapped ring to allow the engagement of the jaws between the tapped ring and the tubular skirt and the screwing of the tapped ring on these jaws.

### TECHNICAL FIELD

This disclosure relates to the technical field of attachment systems for temporarily securing two parts, one of which takes the form of a tube, a rod, or a bar, namely a mast in the general sense.

This disclosure finds applications specifically in the field of non-implantable medical devices where a medical device is to be temporarily attached on a mast of an item of support equipment, such as an intravenous stand or intravenous pole.

A preferred application of the subject of the disclosure relates to the temporary attachment of a display screen of a medical endoscope on an item of support equipment, such as an intravenous stand or intravenous pole.

### BACKGROUND

In the prior art many technical solutions are known for temporarily securing two parts, one of which has the form of a mast. For example, clamp attachment systems are known including two articulated arms under the action of a spring. This attachment system has the advantage of adapting to masts of different diameters. However, this solution presents the difficulty of determining the appropriate tightening force to provide secure attachment of a relatively weighty part on the mast, without impeding operations of mounting and dismounting of the part on the mast.

Screw attachment systems are also known including a hoop in which the mast is engaged. One of the arms of the hoop is equipped with a screw, the tightening of which on the mast makes it possible to attach the part fitted with the hoop, as described for example by the patent application CN 206151751. The drawback of this screw attachment system is its low tightening force which makes it impossible to ensure a secure and effective attachment of a relatively weighty part on the mast. Moreover, the manipulations for adjusting the orientation around the mast require the use of both hands.

In the field of the attachment of a vertebral attachment rod on pedicular screws, the patent application US 2003/149431 describes a system including a spinal attachment rod which is coupled to a vertebra by way of a pedicle screw anchored in a vertebra. This system includes a tubular body receiving, at one end the head of the pedicle screw while the other end is externally provided with a thread to receive an external nut for locking the inserted rod into a diametrical opening arranged in the tubular body. The spinal attachment rod is locked in position with respect to the tubular body using an internal nut cooperating with a tapping of the tubular body.

In the same way, the patent U.S. Pat. No. 6,843,791 describes a spinal attachment system including a spinal attachment rod which is coupled to a vertebra by way of a pedicle screwed anchored in a vertebra. This system includes a tubular body receiving, at one end, the head of the pedicle screw while the other end is externally provided with a thread to receive a nut for locking the rod inserted into a diametrical opening arranged in the tubular body. The locking nut also includes a rotary threaded internal part screwed onto the tubular body to lock the spinal attachment rod.

Document EP 1 348 388 also describes a spinal attachment system including a spinal attachment rod which is coupled to a vertebra by way of a pedicle screwed anchored in a vertebra. This system includes a tubular body having a diametrical housing for the mounting of the rod assembled using a locking nut screwed onto the tubular body.

The patent application US 2014/018867 also describes a pedicle screw including a head having a diametrical housing for the mounting of an attachment rod assembled using a locking nut including a central filling element and screwed onto the external part of the head of the screw.

The patent application CN 105496540 also describes a spinal attachment system including a spinal attachment rod which is coupled to a vertebra by way of a pedicle screwed anchored in a vertebra. This system includes a tubular body having a diametrical housing for the mounting of the rod assembled using a locking nut screwed onto the tubular body and including an internal part bearing on the rod.

In the field of non-implantable medical devices, the analysis of the state of the art leads to the observation that there is a need to have access to an attachment system adapting to masts of different diameters, having sufficient tightening force to lock a weighty part in a stable position while allowing for easy adjustment of the orientation about the mast.

Such a need arises particularly in the medical field where a non-implantable medical device is to be temporarily attached on a mast of an item of support equipment, such as an intravenous stand or an intravenous pole.

### SUMMARY

This disclosure therefore aims to remedy the drawbacks of the state of the art by proposing a new item of equipment provided with a removable attachment system designed to adapt to masts of different diameters, having a sufficient tightening force to lock a weighty part in a fixed position while allowing for easy adjustment of the orientation about the mast.

To achieve such an objective, the item of equipment includes a system for attaching a non-implantable medical device on a mast, the attachment system includes a body equipped with the medical device and from which protrude face-to-face two jaws delimiting together an open engagement housing for the mast ending, at the level of the body, in a bottom which gradually decreases along a frustoconical portion to adapt to masts of different diameters, both jaws being externally threaded by a thread and having free ends from which is engaged a tapped locking wheel including a tapped ring and a tubular support skirt for the jaws bearing with the opposite surface of the jaws to that provided with the thread, this tubular skirt being secured to the tapped ring and extending coaxially inside the tapped ring to allow the engagement of the jaws between the tapped ring and the tubular skirt and the screwing of the tapped ring on these jaws, in order to provide the tightening of the mast between the bottom of the housing and the tapped locking wheel.

According to a preferred exemplary embodiment, the jaws are embodied by cylinder portions each extending along an angular interval between 30° and 60°.

According to an advantageous variant embodiment, the tapped ring has a length greater than the length of the tubular skirt to allow the axial engagement, without rotation, of the tapped locking wheel on the jaws.

Advantageously, the tapped ring is equipped with a locking flange extending externally to one of the ends of the tapped ring in the form of an annular disk, intended to abut the mast.

According to an advantageous embodiment feature, the tapped locking wheel externally includes a groove bordered on either side by a locking flange and by a stop flange to form a housing for winding a cable.

According to a variant embodiment, the body is equipped with a removable system for attaching a non-implantable medical device.

For example, the non-implantable medical device is mounted on the body along an articulation allowing for the relative rotation of the body with respect to the non-implantable medical device, along the articulation axis.

Advantageously, the body is provided with a stop limiting the pivoting about the articulation axis of the non-implantable medical device.

According to the disclosure, the item of equipment includes an item of stand equipment having a mast on which is mounted an attachment system in accordance with the disclosure.

According to a preferred application, the non-implantable medical device is a display screen intended to be connected to a medical endoscope.

Various other features emerge from the description given below with reference to the appended drawings which show, by way of non-limiting example, forms of embodiment of the subject of the disclosure.

### BRIEF DESCRIPTION OF THE DRAWINGS

FIG. 1 is a side view of an exemplary embodiment of an item of equipment provided with a removable attachment system in accordance with the disclosure, providing the mounting of a non-implantable medical device on an item of support equipment.

FIG. 2 is a perspective view of an item of equipment provided with a removable attachment system in accordance with the disclosure.

FIG. 3 is a perspective view of a part of the item of equipment provided with the removable attachment system in accordance with the disclosure.

FIG. 4 is a section view of the item of equipment provided with the removable attachment system in accordance with the disclosure.

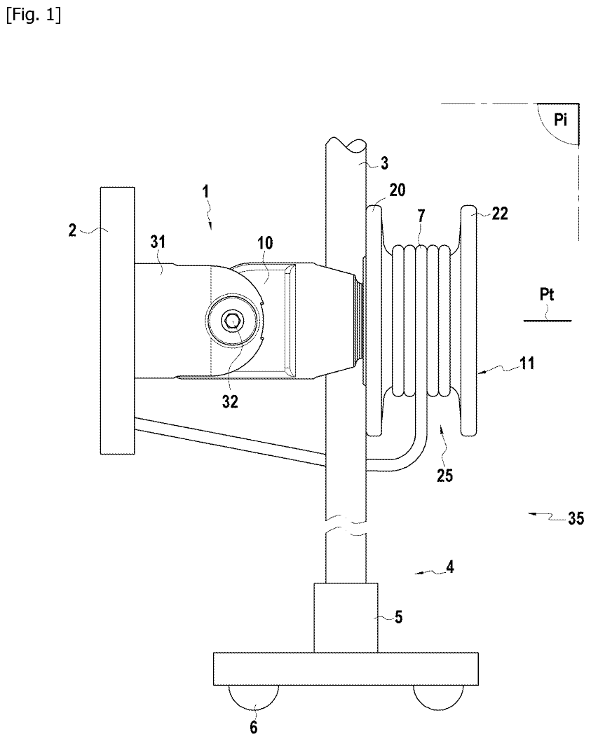

### DETAILED DESCRIPTION

As shown more specifically in the Figures, the subject of the disclosure relates to an item of equipment provided with a removable attachment system 1 providing a dismountable assembly of a part 2 on a mast 3 in the general sense such as a tube, a rod, a column or a bar for example. The mast 3 is part of a support equipment 4 of any type known per se. In a preferred application, the support equipment 4 falls within the medical field. By way of example, the support equipment 4 includes a fixed or movable base 5 from which the mast 3 rises vertically. Advantageously, the support equipment 4 is movable such that the base 5 includes wheels 6.

The support equipment 4 can thus be a medical accessory commonly known as an intravenous stand or intravenous pole. Of course, the support equipment 4 can be embodied differently such as in the form of an item of furniture, a bed or a trolley equipped with a mast extending over a more or less limited height.

The removable attachment system 1 in accordance with the disclosure is intended to be temporarily mounted on the mast 3 of the support equipment 4. In accordance with the disclosure, the removable attachment system 1 is provided as a part 2, of a medical device not implantable in the human body. This non-implantable medical device 2 is thus accessible throughout its use while being able to be moved along the mast and detached from the mast. For example, the non-implantable medical device 2 is a medical monitor or advantageously, a display screen intended to be connected to a medical endoscope. This display screen can thus be fixed temporarily on the support equipment 4 (intravenous stand or intravenous pole for example) particularly during the use of the medical endoscope to which the display screen 2 is connected which conventionally includes at least one cable 7.

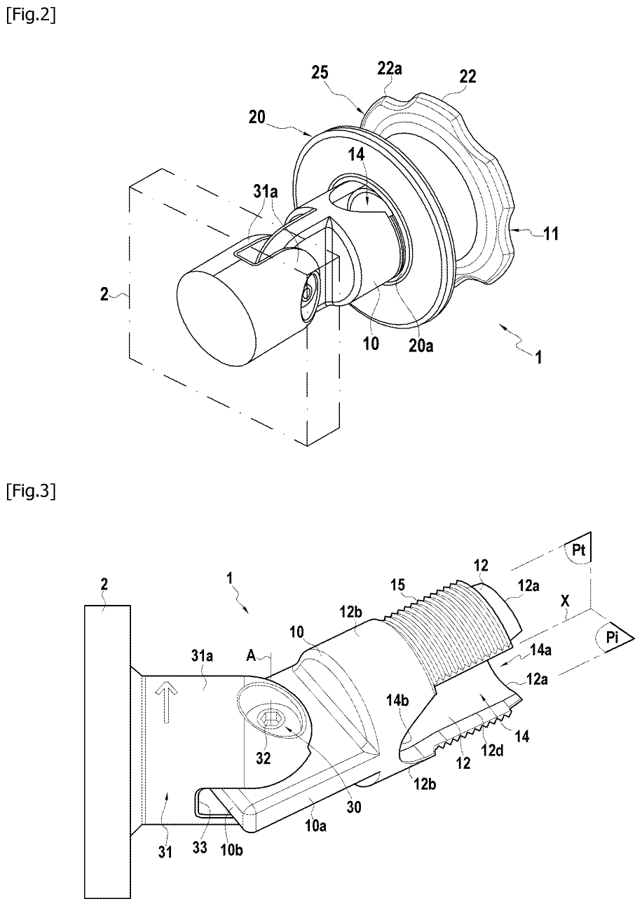

As shown more specifically in FIGS. 2 to 4, the removable attachment system 1 is of screw-nut type such that it is made out of two independent parts. The removable attachment system 1 thus includes a body 10 forming the screw part and equipped with the non-implantable medical device 2 and a nut part embodied in the form of a tapped locking wheel 11 intended to be screwed onto the body 10. The body 10 includes two flaps or jaws 12 extending in protrusion from the body 10 and face-to-face to delimit together an open engagement housing 14 for the mast 3. Each jaw 12 has a free end 12a and at the opposite end, a connection end 12b to the body 10.

The two jaws 12 have an external surface circumscribed in a cylinder of circular section of longitudinal axis X and provided with a thread 15. The jaws 12 thus have a threaded external surface. Of course, the thread 15 embodied on the jaws 12 is suitable for ensuring the screwing of the tapped locking wheel 11. As shown more specifically in FIG. 3, the two jaws 12 are two opposite segments of a tube externally provided with the thread 15 and opened by a diametrical slot arranged from its free end in order to embody the open housing 14. The jaws 12 are embodied by two symmetrical portions of a cylinder each extending along an angular interval between 30° and 60° for example. The two jaws 12 thus extend symmetrically on either side of an insertion plane Pi.

The two jaws 12 together delimit a housing 14 opening by a passage 14a at the level of the free ends 12a and ending in a bottom 14b at the level of the body 10. As will be explained in the rest of the description, the bottom 14b serves as a stop to the mast 3 which is interposed between the bottom 14b and the tapped locking wheel 11. Thus, the thread 15 can advantageously extend in recess from the bottom 14b of the open housing 14.

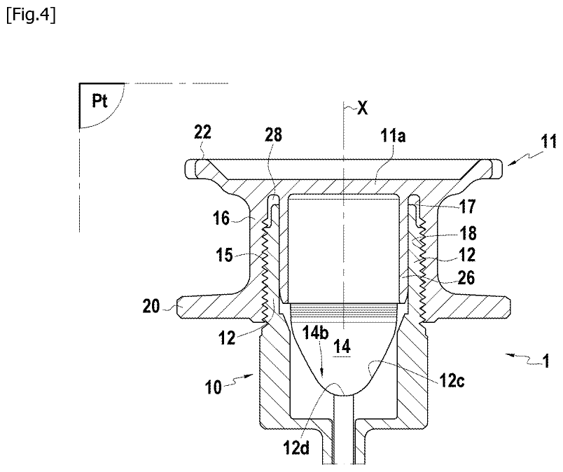

According to an advantageous embodiment feature, the bottom 14b of the open housing 14 has in a transverse plane Pt perpendicular to the insertion plane Pi, a section which gradually increases in the direction of the free ends 12a of the jaws 12 as appears clearly in FIG. 4. Thus, the two jaws 12 have in the transverse plane Pt, a section 12c which increases in the direction of the body 10 to delimit the bottom 14b. The open housing 14 thus has in the transverse plane Pt, a constant section on the basis of the free ends 12a of the jaws over for example ⅔ of its length and a section forming the bottom 14b and which gradually decreases along a frustoconical portion 12c to end for example in a rounded portion 12d. The bottom 14b is thus suitable, by virtue of its frustoconical portion 12c which gradually increases in the direction of the free ends 12a of the jaws 12, to act as a stop for masts 3 of different diameters. The removable attachment system 1 in accordance with the disclosure can be mounted on a mast 3, the widest diameter of which corresponds to the width of the open housing 14 caught between the jaws in the transverse plane Pt and along a direction perpendicular to the longitudinal axis X. For masts 3 of smaller diameter, the masts engage more or less deeply in the frustoconical part 12c of the open housing 14.

As shown more specifically in FIG. 4, the tapped locking wheel 11 includes a tapped ring 16 having a longitudinal axis X. For this purpose, this ring 16 includes an internal tubular face 17 equipped with a thread 18 intended to cooperate with the thread 15 of the jaws. This tapped locking wheel 11 is engaged once the mast 3 is engaged in the open housing 14, from the free ends 12b of the jaws to be screwed onto these jaws 12, in order to provide the tightening of the mast 3 between the bottom 14b of the housing and the tapped locking wheel 11.

Preferably, the tapped locking wheel 11 is equipped with a locking flange 20 protruding externally from one of the ends of the tapped ring 16 in the form of an annular disk centered along the longitudinal axis X and having a central hole 20a to allow for the engagement of the tapped locking wheel on the jaws 12. When screwing the tapped locking wheel 11, the locking flange 20 abuts the mast 3 contributing to the effective tightening of the body 10 on the mast 3.

According to another advantageous embodiment feature, the tapped locking wheel 11 includes a stop flange 22 extending externally to the end of the tapped ring 16, opposite the one provided with the locking flange 20. This stop flange 22 is embodied in the form of a disk centered along the longitudinal axis X and provided at its circumference with notches 22a facilitating the rotational maneuvering of the tapped locking wheel 11. Advantageously, the stop flange 22 and the locking flange 20 together delimit a groove 25 to form a housing for winding a cable 7.

According to an advantageous embodiment feature, the tapped locking wheel 11 includes a tubular support skirt 26 for the jaws 12, extending coaxially to the tapped ring 16 and inside the tapped ring. This tubular skirt 26 is secured to the tapped ring 16 to form a single part. As shown in FIG. 4, the tapped locking wheel 11 includes a transverse face 11a from which the tubular skirt 26 and the tapped ring 16 extend concentrically in such a way as to together delimit an annular space 28 allowing the engagement of the jaws 12 between the tapped ring 16 and the tubular skirt 26. The transverse face 11a is for example a closed face advantageously protruding with respect to the tapped ring 16 to form the stop flange 22.

It must be understood that this tubular skirt 26 is positioned in such a way as to come into contact with the smooth internal surface of the jaws 12 opposite that provided with the thread 15. As shown in the drawings, the tubular skirt 26 has a smooth external surface intended to come into contact with the smooth internal surface of the jaws 12. Thus, the two jaws 12 bear in a position holding them parallel to one another. The tubular skirt 26 thus provides, during the tightening operation, the proper cooperation between the threading 15 of the two jaws 12 and the threading 18 of the tapped locking wheel 11. The tubular skirt 26 thus avoids the bending of the jaws 12 able to alter the cooperation between the threads 15, 18.

According to the example illustrated on the drawings, the tubular skirt 26 and the tapped ring 16 have substantially the same length along the longitudinal axis X. According to a variant embodiment, it should be noted that the tapped ring 16 can have a length greater than the length of the tubular skirt 26 to allow the axial engagement, without rotation, of the tapped locking wheel on the jaws 12, over a limited length. For example, the tapped ring 16 has a length which is double the length of the tubular skirt 26.

The implementation of the removable attachment system 1 in accordance with the disclosure follows on directly from the previous description. When the body 10 is separated from the tapped locking wheel 11, the mast 3 can be engaged in the open housing 14 between the jaws 12. The tapped locking wheel 11 can then be screwed from the free ends of the jaws 12. The tapped locking wheel 11 is screwed until the mast 3 becomes locked between the bottom of the housing 14 and the tapped locking wheel 11.

It should be noted that the removable attachment system 1 in accordance with the disclosure can be implemented without the body 10 becoming separated from the tapped locking wheel 11. In this case, the removable attachment system 1 is disposed such that the mast 3 can be threaded into the part of the open housing 14 which is accessible between the body 10 and the tapped locking wheel 11.

The removable attachment system 1 in accordance with the disclosure thus makes it possible to fix a non-implantable medical device 2 quickly, easily and securely on the mast 3 of the support equipment 4, such a mast being able to be of different diameters. This removable attachment system 1 also allows for easy adjustment of the non-implantable medical device 2 both in height along the mast 3 and in azimuth about the mast 3.

It should be understood that the mast 3 becomes locked between the bottom 14b of the housing 14 and the tapped locking wheel 11. Note that an elastomer material can be interposed between the mast 3 and the bottom 14b of the housing and/or between the mast 3 and the tapped locking wheel 11 and more precisely, the locking flange 20. This elastomer material, which can be integrated or attached, is able to reinforce the adhesion of the attachment system to the mast 3.

According to an exemplary embodiment, the body 10 is rigidly fixed to the non-implantable medical device 2 using a full coupling. According to an exemplary embodiment illustrated in the drawings, the non-implantable medical device 2 is mounted on the body 10 along an articulation 30 permitting the relative rotation of the non-implantable medical device 2 with respect to the body 10, about an articulation axis A. For example, the articulation axis A extends along a direction perpendicular to the insertion plane Pi such that the non-implantable medical device 2 can be oriented in elevation, which makes it possible to adjust the inclination of the non-implantable medical device 2 with respect to the horizontal plane when the non-implantable medical device 2 is mounted on a vertical mast 3. Such a variant is advantageous when the non-implantable medical device 2 is a display screen.

The articulation 30 is embodied in any appropriate manner such as in the form of a hinge including a plate 10a protruding from the body 10 and engaged between the arms 31a of a yoke 31 attached to the non-implantable medical device 2. The yoke 31 and body 10 are assembled together by a pin 32 defining the articulation axis A and traversing the arms 31a and the plate 10a to permit the rotation of the non-implantable medical device 2 about the articulation axis A. According to an advantageous embodiment feature, the articulation 30 between the yoke 31 and body 10 is embodied with a predetermined adhesion such that the angular position which is imposed between the yoke and the body 10 is preserved. In other words, the friction between the arms 31a and the plate 10a is such that in the absence of application of a force on the non-implantable medical device 2, the non-implantable medical device 2 remains in a stable position with the orientation it has been given with respect to the body 10. Of course, the non-implantable medical device 2 is able to occupy different stable positions corresponding to several orientations in elevation.

According to an advantageous embodiment feature, the body 10 is provided with a stop 10b limiting the pivoting of the non-implantable medical device 2 along the articulation axis A. In the illustrated example, the plate 10a of the body 10 includes a free end edge 10b engaged between the arms 31a of the yoke 31 and against which the bottom 33 of the yoke is intended to abut for a predetermined angular position between the body 10 and the yoke 31. This stop position for which the bottom 33 of the yoke is abutting the free end edge 10b of the body 10 determines the maximum angular position that can be taken up by the non-implantable medical device 2 with respect to the body 10.

According to another non-illustrated variant embodiment, the body 10 is equipped with a removable or dismountable system for attaching the non-implantable medical device 2. In other terms, this removable attachment system makes it possible to temporarily join the non-implantable medical device 2 to the body, or even to the yoke 31. Such a system is suitable for easy mounting and dismounting of the non-implantable medical device 2 with respect to the body 10 or the yoke 31. This removable attachment system can be embodied in any appropriate manner such as in the form of complementary sliders arranged on the non-implantable medical device 2 and the body 10 or else the yoke 31.

The subject of the disclosure also relates to an item 35 of equipment including support equipment 4 having a mast 3 on which is mounted an attachment system 1 in accordance with the disclosure. Such an attachment system 1 has a body 10 on which is mounted a non-implantable medical device 2 which according to a preferred application is a display screen intended to be connected to a medical endoscope.

1-10. (canceled)

11. An item of equipment including a system for attaching a non-implantable medical device on a mast, the attachment system including a body equipped with the medical device and from which protrude face-to-face two jaws together delimiting an open engagement housing for the mast ending, at the level of the body, in a bottom which gradually decreases along a frustoconical portion to adapt to masts of different diameters, both jaws being externally threaded by a thread and having free ends from which is engaged a tapped locking wheel including a tapped ring and a tubular support skirt for the jaws bearing with the opposite surface of the jaws to that provided with the thread, this tubular skirt being secured to the tapped ring and extending coaxially inside the tapped ring to allow the engagement of the jaws between the tapped ring and the tubular skirt and the screwing of the tapped ring on these jaws, in order to provide the tightening of the mast between the bottom of the housing and the tapped locking wheel.

12. The item of equipment of claim 11, wherein the jaws are embodied by cylinder portions each extending along an angular interval between 30° and 60°.

13. The item of equipment of claim 11, wherein the tapped ring has a length greater than the length of the tubular skirt, to allow the axial engagement, without rotation, of the tapped locking wheel on the jaws.

14. The item of equipment of claim 11, wherein the tapped ring is equipped with a locking flange extending externally to one of the ends of the tapped ring in the form of an annular disk, intended to abut the mast.

15. The item of equipment of claim 11, wherein the tapped locking wheel externally includes a groove bordered on either side by a locking flange and by a stop flange to form a housing for winding a cable.

16. The item of equipment of claim 11, wherein the body is equipped with a removable system for attaching a non-implantable medical device.

17. The item of equipment of claim 11, wherein the medical device is mounted on the body along an articulation allowing for the relative rotation of the body with respect to the non-implantable medical device, along the articulation axis.

18. The item of equipment of claim 17, wherein the body is provided with a stop limiting the pivoting about the articulation axis of the non-implantable medical device.

19. The item of equipment of claim 11, wherein it includes a support equipment having a mast on which is mounted an attachment system.

20. The item of equipment of claim 11, wherein the non-implantable medical device is a display screen intended to be connected to a medical endoscope.
